(19) United States
(12) Patent Application Publication
(10) Pub. No.: US 2011/0136711 A1
(43) Pub. Date: Jun. 9, 2011

(54) HIGHLY OVERBASED MAGNESIUM ALKYTOLUENE SULFONATES

(75) Inventors: Ping Wang, Fremont, CA (US); Eugene Spala, Fairfield, CA (US)

(73) Assignee: Chevron Oronite Company LLC

(21) Appl. No.: 12/629,999

(22) Filed: Dec. 3, 2009

(51) Int. Cl. C10M 159/24; C07C 307/00

(52) U.S. Cl. 508/391; 558/56

(57) ABSTRACT

A process for preparing a highly overbased magnesium alkyltoluene sulfonates comprising: (A) reacting at least one oil soluble alkyltoluene sulfonic acid and at least one source of magnesium, in the presence of a mixture comprising: (i) at least one hydrocarbon solvent; and (ii) at least one low molecular weight alcohol; (B) contacting the reaction product of (A) with at least one promoter and water; (C) contacting the product of (B) with an overbasing acid; and (D) heating the reaction product of (C) to a temperature greater than the distillation temperature of the hydrocarbon solvent, the low molecular weight alcohol, and the water to distill the hydrocarbon solvent, the low molecular weight alcohol, and the water.

### FIELD OF THE INVENTION

The present invention is directed to an improved process for making highly overbased magnesium alkyltoluene sulfonates. The present invention is also directed to the products made by that process. The present invention is also directed to a lubricating oil composition and a lubricating oil additive concentrate containing the highly overbased magnesium alkyltoluene sulfonates made by the improved process.

### BACKGROUND OF THE INVENTION

Generally, overbased magnesium sulfonates are more difficult to prepare than corresponding overbased calcium sulfonates. Processes for preparing overbased magnesium sulfonates often require special reaction conditions and the incorporation of additional substances into the mixture to be carbonated such as low molecular alcohols, various promoters, and water. In addition, the processes for preparing highly overbased magnesium sulfonates often result in undesirably high levels of hard sediment and/or gelatinous material known as post-carbonation sediment (PCS) which is formed during the overbasing process. It is desirable that overbased materials used as additives in lubricating oil compositions be clear liquids and be free of sediment.

### BACKGROUND ART

Certain methods of making highly overbased magnesium sulfonates are known in the art. Research Disclosure No. 31826 (October 1990), available at www.researchdisclosure.com, discloses overbased magnesium sulfonates prepared by carbonation of a reaction mixture comprising an alkyl toluene or xylene sulfonic acid and an excess of a magnesium compound typically with a hydrocarbon solvent and usually in the presence of a promoter. The alkyl group preferably contains 10 to 30 carbon atoms, most preferably 18 to 24 carbon atoms.

Gergel, et al., U.S. Pat. No. 3,629,109 discloses basic magnesium salts of organic acids prepared by a process which comprises contacting at least one acidic material with at least one oil-soluble organic acid or suitable derivative thereof and at least one basically reacting magnesium compound in the presence of water or a combination of at least one alcohol and water.

Cleverly et al., U.S. Pat. No. 5,534,168 discloses the use of magnesium oxide of relatively low reactivity in the process for the production of overbased magnesium sulfonates, together with the introduction of water and an alcohol during the carbonation step, which provide high base number products with low post-carbonation sediment (PCS) values and which can be purified by rapid filtration.

Moulin et al., WO 97/14774 discloses the use of a low molecular weight sulfonic acid, or magnesium salt thereof that is at least partially water-soluble in the process to prepare highly overbased, high molecular weight magnesium sulfonates with low post-carbonation sediment.

Muir, U.S. Pat. No. 4,617,135 discloses a one step process for production of highly overbased magnesium sulfonates with minimal sediment by using a combination of specified first and second promoters.

Muir, U.S. Pat. No. 4,647,387 discloses an oil composition which comprises (a) an engine oil or lubricating oil, and (b) an overbased magnesium sulfonate, and is practically free of water-tolerance additives added after sulfonate formation. Water tolerance is provided by a succinic anhydride promoter reaction product present during the formation of component (b).

Cleverly et al., EP-A-323 088 discloses a process for the preparation of overbased magnesium sulfonate in which the reaction mixture for carbonation contains an effective amount of an ashless dispersant. The overbased magnesium sulfonates have reduced interactions, sedimentation and haze.

### SUMMARY OF THE INVENTION

The present invention is directed to the discovery of an improved method of making highly overbased magnesium alkyltoluene sulfonates. The present invention is also directed to the products made by that process. The present invention is also directed to a lubricating oil composition and a lubricating oil additive concentrate containing the highly overbased magnesium alkyltoluene sulfonates made by the improved process.

Accordingly, in its broadest embodiment, the present invention is directed to a process for preparing a highly overbased magnesium alkyltoluene sulfonates comprising:

(a) reacting at least one oil soluble alkyltoluene sulfonic acid and at least one source of magnesium, in the presence of a mixture comprising:
(i) at least one hydrocarbon solvent; and
   (ii) at least one low molecular weight alcohol;
(b) contacting the reaction product of (a) with at least one promoter and water;
(c) contacting the product of (b) with an overbasing acid; and
(d) heating the reaction product of (c) to a temperature greater than the distillation temperature of the hydrocarbon solvent, the low molecular weight alcohol, and the water to distill the hydrocarbon solvent, the low molecular weight alcohol, and the water.

A further embodiment of the present invention is directed to a highly overbased magnesium alkyltoluene sulfonate prepared by the process of the present invention.

A further embodiment of the present invention is directed to a lubricating oil composition comprising an oil of lubricating viscosity and a minor amount of a highly overbased magnesium alkyltoluene sulfonate prepared by the process of the present invention.

A further embodiment of the present invention is directed to a lubricating oil concentrate comprising from about 90 wt. % to about 10 wt. % of an organic liquid diluent and from about 10 wt. % to about 90 wt. % of a highly overbased magnesium alkyltoluene sulfonate prepared by the process of the present invention.

### DETAILED DESCRIPTION OF THE INVENTION

While the invention is susceptible to various modifications and alternative forms, specific embodiments thereof have been shown by way of example and are herein described in detail. It should be understood, however, that the description herein of specific embodiments is not intended to limit the invention to the particular forms disclosed, but on the contrary, the intention is to cover all modifications, equivalents, and alternatives falling within the spirit and scope of the invention as defined by the appended claims.

The term “Total Base Number” or “TBN” refers to the amount of base equivalent to milligrams of KOH in 1 gram of sample. Thus, higher TBN numbers reflect more alkaline products and therefore a greater alkalinity reserve. For the purposes of this invention, TBN is determined by ASTM Test No. D2896.

The term “overbased” refers to sulfonates containing an amount of metal in excess of that required to react with the sulfonic acid from which the sulfonate is obtained.

The term “highly overbased” refers to sulfonates having a TBN of about 300 or greater.

Each and every numerical range and/or value, regardless whether it appears immediately or closely following the term “about,” should be understood to encompass those variations that may represent the range of experimental error that occur in any measurement.

The term “post-carbonation sediment” or “PCS” refers to the proportion of sediment in the reaction mixture after the carbonation step is completed. PCS is expressed as volume % based on the volume of the reaction mixture.

The term “oil of lubricating viscosity” as used herein refers to base oils which may be mineral oil or synthetic oils of lubricating viscosity and preferably useful in the crankcase of an internal combustion engine. Crankcase lubricating oils ordinarily have a viscosity of about 1300 centistokes at −17.8° C. to 22.7 centistokes at 98.9° C. The lubricating oils may be derived from synthetic or natural sources. Mineral oil for use as the base oil in this invention includes paraffinic, naphthenic and other oils that are ordinarily used in lubricating oil compositions. Synthetic oils include hydrocarbon synthetic oils, synthetic esters and Fischer-Tropsch derived base oil. Useful synthetic hydrocarbon oils include liquid polymers of alpha-olefins having the proper viscosity. Especially useful are the hydrogenated liquid oligomers of C₆ to C₁₂ alpha-olefins such as 1-decene trimer. Similarly, alkyl benzenes of proper viscosity, such as didodecyl benzene, may be used. Useful synthetic esters include the esters of both mono-carboxylic acids and polycarboxylic acids as well as mono-hydroxy alkanols and polyols. Typical examples are didodecyl adipate, pentaerthritol tetracapoate, di-2-ethylhexyl adipate, di-laurylsebacate and the like. Complex esters prepared from mixtures of mono- and di-carboxylic acid and mono- and di-hydroxy alkanols can also be used. Blends of hydrocarbon oils and synthetic oils may also be used. For example, blends of 10 wt. % to 25 wt. % hydrogenated 1-decene trimer with 75 wt. % to 90 wt. % 683 centistokes at 37.8° C. mineral oil gives an excellent oil base.

### Process for Preparing a Highly Overbased Magnesium Alkyltoluene Sulfonate

The present invention involves an improved process for preparing a highly overbased magnesium alkyltoluene sulfonate.

The process for preparing a highly overbased magnesium alkyltoluene sulfonate comprises:
(a) reacting at least one oil soluble alkyltoluene sulfonic acid and at least one source of magnesium, in the presence of a mixture comprising:
   (i) at least one hydrocarbon solvent; and
   (ii) at least one low molecular weight alcohol;
(b) contacting the reaction product of (a) with at least one promoter and water;
(c) contacting the product of (b) with an overbasing acid; and
(d) heating the reaction product of (c) to a temperature greater than the distillation temperature of the hydrocarbon solvent, the low molecular weight alcohol, and the water to distill the hydrocarbon solvent, the low molecular weight alcohol, and the water.

### Alkyltoluene Sulfonic Acid

In the present invention, an oil soluble alkyltoluene sulfonic acid is used in the process for preparing a highly overbased magnesium alkyltoluene sulfonate. The alkyltoluene sulfonic acid may be derived from sulfonating an alkyltoluene, such as linear alkyltoluene or branched alkyltoluene using various known sulfonating agents, such as, for example, sulfuric acid, sulfur trioxide, chlorosulfonic acid or sulfamic acid. Other conventional methods, such as the SO₃/Air Thin Film Sulfonation method may also be applied, wherein the alkyltoluene precursor is mixed with a SO₃/air falling film made by Chemithon (Seattle, Wash.) or Ballestra (Milan, Italy).

The alkyltoluene may be derived from the alkylation of toluene with a linear olefin, which comprises at least 16 carbon atoms, in the presence of a Lewis Acid. Preferably, the olefin is a normal alpha olefin that has from about 18 to about 28 carbon atoms. More preferably, the olefin is a normal alpha olefin that has from about 20 to about 24 carbon atoms. Alkylated aromatics, the process of which is well known in the art, may be derived from numerous processes including, but not limited to, the processes disclosed in U.S. Patent Application Publication Nos. US 2005/0202954, US 2005/0203323, and US 2005/0203322.

In addition, branched alkyltoluenes may be prepared by the alkylation of toluene with a branched-chain olefin. The branched chain olefin may be prepared the isomerization of a normal alpha olefin having at least 16 carbon atoms. The normal alpha olefin may isomerized prior to, during, or after the alkylation step, but is preferably isomerized prior to the alkylation step. Methods of isomerizing olefins are known in the art. Preferably, the branched chain olefin is derived from the isomerization of normal alpha olefin that has from about 18 to about 28 carbon atoms. More preferably, the branched chain olefin is derived from the isomerization of a normal alpha olefin that has from about 20 to about 24 carbon atoms. The branched chain olefin may also be derived from an oligomer of propylene or butene comprising at least 9 carbon atoms, preferably from about 9 to 40 carbon atoms, more preferably from about 9 to 24 carbon atoms and most preferably from about 10 to 18 carbon atoms.

Preferably, the alkyltoluene sulfonic acid is obtained by the sulfonation of a mixture of primarily mono alkyltoluene which is obtained from the alkylation of toluene by a mixture of normal alpha olefins having from about 20 to about 24 carbon atoms.

### Source of Magnesium

At least one source of magnesium is also reacted with the aforementioned oil soluble alkyltoluene sulfonic acid in the presence of a mixture comprising a hydrocarbon solvent and a low molecular weight alcohol. Preferably, the at least one source of magnesium used in the reaction of the present invention is a magnesium oxide, magnesium hydroxide or magnesium alkoxide wherein the alkoxide is derived an alcohol having from 1 to 6 carbon atoms. Preferably, the at least one source of magnesium is magnesium oxide.

### Hydrocarbon Solvent

The hydrocarbon solvent used in the present process may be selected from the group consisting of n-pentane, n-hexane, cyclohexane, n-heptane, n-octane, isooctane, n-decane, benzene, toluene, xylene and mixtures thereof. Preferably, the hydrocarbon solvent is an aromatic solvent and is selected from the group of solvents consisting of xylene, benzene and toluene. The most preferred aromatic solvent is xylene.

### Low Molecular Weight Alcohol

The low molecular weight alcohol must have a boiling point sufficiently low so that it may be easily distilled off after the reaction has occurred. Typically, the low molecular weight alcohol will have from about 1 to about 13 carbon atoms and a molecular weight no higher than about 200. In one embodiment, the low molecular weight alcohol is a low molecular weight monohydric alcohol. In a more preferred embodiment, the low molecular weight monohydric alcohol which may be used in the present process may be selected from the group consisting of C₁ to C₁₃ alcohols and glycol monoethers and monoesters. Preferably, the low molecular weight alcohol is a monohydric alcohol selected from the group consisting of methanol, ethanol, propanol, isooctanol, cyclohexanol, cyclopentanol, isobutyl alcohol, benzyl alcohol, beta-phenyl-ethyl alcohol, 2-ethylhexanol, dodecanol, tridecanol, 2-methylcyclohexanol, monomethyl ether of ethylene glycol, monobutyl ether of ethylene glycol, sec-pentyl alcohol, and tert-butyl alcohol. The most preferred low molecular weight monohydric alcohol is methanol.

### Promoters

Promoters are used to facilitate carbonation, improve filterability or as viscosity agents. The promoter may be an organic monocarboxylic acid, a hydrocarbyl-substituted succinic anhydride or derivative thereof, or mixtures thereof. Suitable organic monocarboxylic acids have from 1 to 24 carbon atoms. The organic monocarboxylic acids may be aliphatic or aromatic, saturated or unsaturated. Preferred organic monocarboxylic acids include formic acid, acetic acid, stearic acid, benzoic acid, salicylic acid, and mixtures thereof. The hydrocarbyl-substituted succinic anhydride or derivative thereof, includes alkyl and alkenyl succinic anhydrides wherein the alkyl or alkenyl group of the succininc anhydride or derivative contains from about 8 to about 70 carbon atoms. Preferred hydrocarbyl-substituted succinic anhydrides include dodecenyl succinic anhydride (DDSA), octadecenyl succinic anhydride (ODSA) and polyisobutenyl succinic anhydride (PIBSA) derived from 1000 MW polyisobutene. Suitable succinic anhydride derivatives include the acids, esters, half-esters, double-esters, and other hydrolyzable derivatives. In one embodiment, the promoter is a mixture of acetic acid and stearic acid. In another embodiment, the promoter is a mixture of acetic acid and DDSA. In another embodiment, the promoter is a mixture of acetic acid and 1000 MW PIBSA.

### Overbasing Acid

The term “overbasing acid,” as used herein, refers to an acid capable of providing an oil-soluble metal sulfonate with greater than a stoichiometric amount of metal to sulfonic acid. The most common overbasing acid is carbon dioxide; other overbasing acids include sulfur dioxide and sulfur trioxide. The acid itself may be part of the overbasing process, or alternatively a source of an overbasing acid such as ethylene carbonate may be used to introduce the overbasing acid. The most preferred acid is carbon dioxide and the treatment with overbasing acid will in general be referred to as “carbonation.” Unless the context clearly requires otherwise, it will be understood that references herein to carbonation include references to treatment with other overbasing acids.

### Process and Diluent Oils

If the magnesium sulfonate is viscous, an inert liquid medium may be employed to reduce the viscosity. The inert liquid medium can also serve to disperse the product and to facilitate mixing of the ingredients. A preferred inert liquid medium is a base oil of lubricating viscosity.

Foam inhibitors and other processing aids may also be added.

In a typical process of the present invention, a hydrocarbon solvent is first premixed with a low molecular weight alcohol and a source of magnesium. The source of magnesium may be added as a single charge in the pre-mix or as multiple charges in the pre-mix and in the subsequent carbonation step. Generally this premixing is done near ambient temperatures, i.e. at about 15-40° C. Sulfonic acid is next added with agitation. Typically, sulfonic acid is added over a period of time wherein the temperature ranges from about 20° C. to about 40° C. The mixture is held for approximately 5 to 20 minutes at about 40° C. to ensure that the sulfonic acid is adequately neutralized by the source of magnesium to make a neutral magnesium sulfonate.

Prior to carbonation, at least the promoter and the water are added with agitation to the mixture containing the neutralized sulfonate while maintaining a temperature of about 40° C.

Carbonation is carried out at a constant temperature, typically, at about 40° C. Carbonation may be carried out either at a pressure below atmospheric pressure, at atmospheric pressure, or at a pressure above atmospheric pressure. Preferably, carbonation is carried out at atmospheric pressure.

Upon completion of carbonation, the reaction mixture is heated to a temperature of from about 125° C. to about 140° C. Typically, a diluent oil, such as 100N oil, will be added to the mixture and any solids are subsequently removed by conventional methods, such as centrifugation or filtration. Finally, the hydrocarbon solvent, low molecular weight alcohol and the water are removed by distillation.

The present invention is also directed to a product that results from the process described herein. The resulting overbased magnesium alkyltoluene sulfonates have a TBN of about 300 or greater, preferably about 350 or higher, most preferably about 400 or greater. The magnesium content of the resulting magnesium alkyltoluene sulfonates is from about 8.0 wt. % to about 11.0 wt. % preferably from about 9.0 wt. % to about 10.0 wt. %, more preferably from about 9.2 wt. % to about 9.8 wt. %.

The present invention is also directed to a lubricating oil composition comprising a major amount of an oil of lubricating viscosity and a minor amount of the highly overbased magnesium alkyltoluene sulfonates prepared by the process of the present invention. Typically the lubricating oil composition will contain highly overbased magnesium alkyltoluene sulfonates prepared by the process of the present invention in the range of from about 0.01 wt. % to about 10 weight percent, preferably in the range of from about 0.1 wt. % to about 5 wt. % and more preferably in the range of from about 0.3 wt. % to about 2 weight percent. The lubricating oil composition will generally contain other additives including detergents (overbased and non-overbased), dispersants, extreme pressure agents, wear inhibitors, rust inhibitors, foam inhibitors, corrosion inhibitors, pour point depressants, antioxidants, zinc dithiophosphates and a variety of other well known additives.

In one embodiment, a lubricating oil composition may contain the following components:

(a) a major amount of oil of lubricating viscosity;

(b) 0.01 wt. % to 10.0 wt. % of at least one highly overbased magnesium alkyltoluene sulfonate prepared by the process of the present invention;

(c) 1.0 wt. % to 10.0 wt. % of at least one borated or non-borated succinimide ashless detergent;

(d) 0.05 wt. % to 0.5 weight percent, as calcium, of at least one calcium sulfonate, phenate or salicylate detergent;

(e) 0.02 wt. % to 0.2 weight percent, as phosphorus, of at least one secondary or mixture of primary and secondary alkyl zinc dithiophosphate;

(f) 0.0 wt. % to 5.0 wt. % of at least one diphenylamine oxidation inhibitor;

(g) 0.0 wt. % to 0.5 wt. % of, as molybdenum, of at least one molybdenum succinimide oxidation inhibitor;

(h) 0.0 wt. % to 5.0 wt. % of at least one partial carboxylic ester or borated ester friction modifier;

(i) 0.0 wt. % to 0.05 wt. % of at least one supplemental anti-wear/extreme pressure agent, such as molybdenum dithiocarbamate;

(j) 0.0 wt. % to 0.1 wt. % of at least one foam inhibitor; and

(k) 0.0 wt. % to 5.0 wt. % of at least one olefin copolymer viscosity index improver.

The present invention is also directed to a lubricating oil additive concentrate. The lubricating oil additive concentrate contains from 90 wt. % to 10 wt. % of an organic liquid diluent and from 10 wt. % to 90 wt. % of the highly overbased magnesium alkyltoluene sulfonate prepared by the process of the present invention. Typically, a neutral oil having a viscosity of about 4 to about 8.5 centistokes (cSt) at 100° C. and preferably about 4 to about 6 cSt at 100° C. will be used as the diluent, though synthetic oils, as well as other organic liquids which are compatible with the additives and finished lubricating oil can also be used.

The invention is further illustrated by the following examples, which set forth particularly advantageous method embodiments. While the examples are provided to illustrate the present invention, they are not intended to limit it.

### EXAMPLES

### Example 1

A 4-necked glass vessel was charged with 75 grams of methanol, 970 grams of xylene and 135 grams of magnesium oxide while stirring. Next, 275 grams of an alkyltoluene sulfonic acid, having a molecular weight of 475 and which was prepared according to the procedure as described in Example A of U.S. Pat. No. 7,479,568, was added to the vessel over 15 minutes while maintaining the temperature in the reaction mixture at about 40° C. followed by addition of 95 grams of water, 50 grams of acetic acid (AA) promoter, and 20 grams of dodecenyl succinic anhydride (DDSA) promoter.

After 15 minutes, carbonation was initiated by introducing into the vessel 22 grams of carbon dioxide over 40 minutes at a temperature of 40° C. Carbonation was continued by introduction into the vessel of 57 grams of carbon dioxide over 66 minutes, of 22 grams of carbon dioxide over 27 minutes, and of an additional 12 grams of carbon dioxide over 37 minutes. The post-carbonation sediment (PCS) was 0.15 vol. %.

The mixture contained within the vessel was taken in stages to a temperature comprised between 40° C. and 132° C. over 2 hours. Then 230 grams of Exxon 100N oil was added to the mixture. The mixture was centrifuged on a Beckman centrifuge and heated to 165° C. while under full vacuum for 15 minutes to eliminate xylene, methanol and water.

### Example 2

A 4-necked glass vessel was charged with 75 grams of methanol, 970 grams of xylene and 275 grams of an alkyltoluene sulfonic acid, having a molecular weight of 475 and which was prepared according to the procedure as described in Example A of U.S. Pat. No. 7,479,568. The temperature of the mixture was held at about 40° C. Next, 135 grams of magnesium oxide was added to the vessel over 15 minutes while maintaining the temperature in the reaction mixture at about 40° C. After 10 additional minutes, 60 grams of water, 35 grams of acetic acid promoter, and 15 grams of stearic acid (SA) promoter were added to the vessel.

After 5 minutes, carbonation was initiated by introducing into the vessel 22 grams of carbon dioxide over 40 minutes at a temperature of 40° C. Carbonation was continued by introduction into the vessel of 57 grams of carbon dioxide over 66 minutes, of 22 grams of carbon dioxide over 27 minutes, and of an additional 12 grams of carbon dioxide over 37 minutes. The PCS was 1.6 vol. %.

The mixture contained within the vessel was taken in stages to a temperature comprised between 40° C. and 132° C. over 90 minutes. Then 230 grams of Exxon 100N oil was added to the mixture. The mixture was centrifuged on a Beckman centrifuge and heated to 200° C. while under full vacuum for 10 minutes to eliminate xylene, methanol and water.

### Example 3

Example 3 was prepared according to the procedure described in Example 2 except that the alkyltoluene sulfonic acid was derived from an alkyltoluene prepared by alkylation of toluene with isomerized C₂₀ to C₂₄ olefins with 23% branching.

A summary of the physical and chemical properties of the overbased magnesium alkyltoluene sulfonates prepared in Examples 1-3 together with two commercial highly overbased magnesium alkylbenzene sulfonates is reported in Table 1.

TABLE 1

| | | | | Comp. | Comp. |
|---|---|---|---|---|---|
| | | | | Sulfonate. | Sulfonate |
| | Ex. 1 | Ex. 2 | Ex. 3 | A* | B** |
| TBN (mg KOH/g) | 411 | 403 | 404 | 397 | 402 |
| Vis. at 100° C. (cSt) | 207 | 102 | 122 | 155 | 107 |
| Mg (%) | 9.6 | 9.5 | 9.1 | 9.3 | 9.2 |
| CO₂ (%) | 8.86 | 9.09 | 8.88 | 8.50 | 10.2 |
| S—Mg (%) | 0.75 | 0.75 | 0.74 | 0.66 | 0.58 |
| Product Sediment | 0.02 | 0.04 | <0.01 | 0.02 | <0.01 |
| (%) | | | | | |

*Highly overbased magnesium alkylbenzene sulfonate available from Chemtura as Hybase ® M-401
**Highly overbased magnesium alkylbenzene sulfonate available from Lubrizol as LZ6465A

### Evaluation of Protection Against Corrosion

### Example 4

A baseline lubricating oil composition was prepared and used for assessing the corrosion performance of the highly overbased magnesium alkyltoluene sulfonates prepared by the process of the present invention against several commercially available highly overbased alkylbenzene sulfonates in the high temperature corrosion bench test (HTCBT). The baseline composition was prepared using the following additives:

(a) 1 wt. % of a borated succinimide;

(b) 6.5 wt. % of a ethylene carbonate post-treated succinimide;

(c) 2.1 wt. % of a high molecular weight polysuccinimide;

(d) 1.75 mM/kg of a low overbased calcium sulfonate;

(e) 23 mM/kg of a high overbased calcium phenate;

(f) 10 mM/kg of a borated calcium sulfonate;

(g) 16 mM/kg of a zinc dialkyldithiophosphate;

(h) 0.3 wt. % of an alkylated diphenylamine;

(i) 0.6 wt. % of a hindered phenolic ester;

(j) 0.25 wt. % of a molybdenum complex;

(k) 5 ppm of a foam inhibitor;

(l) 0.3 wt. % of a pour point depressant;

(m) 2.0 wt. % of a dispersant VII

(n) 2.85 wt. % of a VI improver; and

(o) the balance, a mixture of Group II base oils.

### Example 5

A lubricating oil composition was prepared by top-treating the baseline formulation of Example 4 with 34.5 mM/kg of the highly overbased magnesium alkyltoluene sulfonate as prepared in Example 1.

### Example 6

A lubricating oil composition was prepared by top-treating the baseline formulation of Example 4 with 34.5 mM/kg of the highly overbased magnesium alkyltoluene sulfonate as prepared in Example 2.

### Example 7

A lubricating oil composition was prepared by top-treating the baseline formulation of Example 4 with 34.5 mM/kg of the highly overbased magnesium alkyltoluene sulfonate as prepared in Example 3.

### Example C(Comparative)

A lubricating oil composition was prepared by top-treating the baseline formulation of Example 4 with 34.5 mM/kg of a highly overbased magnesium alkylbenzene sulfonate (Hybase® M-401 from Chemtura), identified as Comparative Sulfonate A in Table 1.

### Example D (Comparative)

A lubricating oil composition was prepared by top-treating the baseline formulation of Example 4 with 34.5 mM/kg of a highly overbased magnesium alkylbenzene sulfonate (LZ6465A from Lubrizol), identified as Comparative Sulfonate B in Table 1.

The corrosion protection of these lubricating oils was determined and compared in a standard ASTM Test No. D6549 (HTCBT) test for their capacity to protect the engine against corrosion. Specifically, four metal coupons including lead, copper, tin and phosphor bronze were immersed in a measured amount of the test oils. Air was passed through the oils at an elevated temperature for a period of time. When the test was completed, the coupons and stressed oils were examined to detect corrosion. Concentrations of lead, copper and tin in the stressed oils are reported in Table 2 below.

TABLE 2

| | | | | Comp. | Comp. | Pass/Fail |
|---|---|---|---|---|---|---|
| | Ex. 5 | Ex. 6 | Ex. 7 | Ex. C | Ex. D | Standard |
| Pb (ppm) | 38 | 48 | 47 | 132 | 60 | ≦100 |
| Cu (ppm) | 4 | 4 | 4 | 6 | 5 | ≦20 |
| Sn (ppm) | 0 | 0 | 0 | 0 | 0 | ≦50 |

The results in Table 2 demonstrate that lubricating oil compositions comprising a highly overbased magnesium alkyltoluene sulfonate have better anti-corrosive capacity than lubricating oil compositions comprising a highly overbased magnesium alkylbenzene sulfonate. Moreover, in at least one instance, the lubricating oil composition comprising a highly overbased alkylbenzene sulfonate failed ASTM Test No. D6549 with regard to lead corrosion.

It is understood that although modifications and variations of the invention can be made without departing from the spirit and scope thereof, only such limitations should be imposed as are indicated in the appended claims.

What is claimed is:

1. A process for preparing a highly overbased magnesium alkyltoluene sulfonate comprising:
(a) reacting at least one oil soluble alkyltoluene sulfonic acid and at least one source of magnesium, in the presence of a mixture comprising:
(i) at least one hydrocarbon solvent; and
(ii) at least one low molecular weight alcohol;
(b) contacting the reaction product of (a) with at least one promoter and water;
(c) contacting the product of (b) with an overbasing acid; and
(d) heating the reaction product of (c) to a temperature greater than the distillation temperature of the hydrocarbon solvent, the low molecular weight alcohol, and the water to distill the hydrocarbon solvent, the low molecular weight alcohol, and the water.

2. The process according to claim 1, wherein the overbased magnesium alkyltoluene sulfonate has a Total Base Number of at least about 300 mg KOH/g.

3. The process according to claim 2, wherein the overbased magnesium alkyltoluene sulfonate has a Total Base Number of at least about 350 mg KOH/g.

4. The process according to claim 1, wherein the oil soluble alkyltoluene sulfonic acid is a linear alkyltoluene sulfonic acid.

5. The process according to claim 4, wherein the linear alkyl group of the linear alkyltoluene sulfonic acid comprises at least 16 carbon atoms.

6. The process according to claim 5, wherein the linear alkyl group of the linear alkyltoluene sulfonic acid comprises about 18 to about 28 carbon atoms.

7. The process according to claim 6, wherein the linear alkyl group of the linear alkyltoluene sulfonic acid comprises about 20 to about 24 carbon atoms.

8. The process according to claim 1, wherein the oil soluble alkyltoluene sulfonic acid is a branched alkyltoluene sulfonic acid.

9. The process according to claim 8, wherein the branched alkyl group of the branched alkyltoluene sulfonic acid is derived from the isomerization of normal alpha olefin having at least 16 carbon atoms.

10. The process according to claim 9, wherein the branched alkyl group of the branched alkyltoluene sulfonic acid is derived from the isomerization of normal alpha olefin having from about 18 to about 28 carbon atoms.

11. The process according to claim 10, wherein the branched alkyl group of the branched alkyltoluene sulfonic acid is derived from the isomerization of normal alpha olefin having from about 20 to about 24 carbon atoms.

12. The process according to claim 1, wherein the source of magnesium is magnesium oxide.

13. The process according to claim 1, wherein the hydrocarbon solvent is xylene.

14. The process according to claim 1, wherein the process wherein the low molecular weight alcohol is a monohydric alcohol.

15. The process according to claim 14, wherein the low molecular weight monohydric alcohol is methanol.

16. The process according to claim 1, wherein the promoter is selected from the group consisting of acetic acid, stearic acid, dodecyl succinic anhydride, polyisobutenyl succinic anhydride and mixtures thereof.

17. The process according to claim 1, wherein the overbasing acid is carbon dioxide.

18. A highly overbased magnesium alkyltoluene sulfonate prepared by the process of claim 1.

19. A lubricating oil composition comprising a major amount of an oil of lubricating viscosity and a minor amount of the highly overbased magnesium alkyltoluene sulfonate prepared by the process of claim 1.

20. A lubricating oil additive concentrate comprising from about 90 wt. % to about 10 wt. % of an organic liquid diluent and from about 10 wt. % to about 90 wt. % of the highly overbased magnesium alkyltoluene sulfonate prepared by the process of claim 1.
